(19) United States
(12) Patent Application Publication
(10) Pub. No.: US 2010/0204708 A1
(43) Pub. Date: Aug. 12, 2010

(54) Carotid guiding catheter (sheath) for carotid percutaneous intervention/stenting with internal fixation device to prevent migration of the Carotid guiding catheter (sheath)

(75) Inventor: Sanjiv Sharma, Bakersfield, CA (US)

Correspondence Address: Sanjiv Sharma, 10806 Ramsgate Way, Bakersfield, CA, 93311, US

(73) Assignee: Sanjiv Sharma, Bakersfield, CA (US)

(21) Appl. No.: 12/710,365

(22) Filed: Feb. 23, 2010

(51) Int. Cl. A61B 17/00

(52) U.S. Cl. 606/108

(57) ABSTRACT

A carotid intervention guiding catheter (sheath) including a central lumen adapted to receive a therapeutic catheter and stent delivery catheter. The catheter includes a soft tip adapted to lodge in the common carotid artery. The distal body of the guiding catheter includes a mechanism to fixate the catheter to the wall of the aorta to prevent sliding of the guiding catheter in and out of the common carotid artery. The mechanism is a collapsible set of wings that come out of the wall of the catheter using a mechanical switch outside the body that deploys the wings and fixate the catheter to the wall of the descending or transverse aorta. The same mechanical switch allows the wings to collapse into the wall of the guiding catheter and allow the catheter to be retrieved out of the body.

### BACKGROUND OF THE INVENTION

This invention provides a stable supporting catheter device for accessing the common carotid artery for performance of carotid intervention, including deployment of distal protection devices and stents into the carotid artery, without the likelihood of the guiding catheter prolapsing out of the common carotid ostium, especially if resistance is encountered in the delivery of the distal embolic protection device or stent into the carotid.

Catheters are used for injecting dye in the performance of carotid angiography for diagnosis; and carotid intervention in order to treat a stenotic lesion in the carotid artery. In these techniques the distal end of a therapeutic catheter is introduced into the aorta by way of the femoral artery. The proximal end of the catheter is then manipulated so its distal end is inserted into the lumen of a selected carotid artery branching off from the aorta. A typical treatment procedure would involve initially inserting a guiding catheter into the aorta and then selectively into the common carotid, followed by the introduction of a suitable therapeutic device, such as an embolic protection device, balloon dilatation catheter, the stent delivery catheter and the retrieval device for removing the embolic protection device. The embolic protection device is passed through the guiding catheter until its distal end portion is positioned past the stenotic lesion in the carotid artery for preventing the embolization of the plaque material into the intracerebral circulation. In all carotid intervention procedures, it is absolutely crucial that the embolic protection device does not move during the passage of additional therapeutic devices like the balloon dilatation catheter, stent delivery catheter and the retrieval catheter; otherwise the risk of embolization through and past the embolic protection device increases substantially. Fixating the guiding catheter and providing more “back up” support with the guiding catheter, after it is fixated to the wall of the aorta by deploying the collapsible wings prevent the forward or backward movement of the embolic protection device.

To assist in advancing the guiding catheter (sheath) for performance of carotid intervention, a guidewire is passed into a diagnostic carotid catheter, like a JB-1 or Simmons catheter and then this guidewire-diagnostic catheter system is telescoped into the guiding catheter (sheath). This combination of guidewire-diagnostic catheter-guiding catheter (sheath) is inserted into the ascending aorta and selective cannulation of the common carotid accomplished by manipulating the proximal end of this system. Then the diagnostic catheter and guidewire are gently withdrawn from inside the guiding catheter (sheath), with particular attention paid to keep stationary the position of the distal end of the guiding catheter (sheath) relative to the carotid (FIG. 1). However, given the built up potential energy and torque forces in the guiding catheter (sheath), there is often a sudden distal movement (“jumping”) of the end of the guiding catheter (sheath) encountered, especially in stiff and calcified aortas.

After positioning of the guiding catheter (sheath) in the common carotid, a distal embolic protection device is passed in through this sheath into the carotid and positioned distal to the stenotic lesion in the internal carotid.

In certain anatomical situations where
the aorta is calcified, or
the aortic arch is type II or type III, or
the carotid artery is tortuous or calcified, or
the carotid lesion is very stenotic or calcified, or
the internal carotid is tortuous distal to the lesion
   resistance is encountered in the passage of the embolic protection device often leading to either unsuccessful delivery of the embolic protection device to the desired location (sufficiently distal to the lesion site), or to a recoil of the guiding catheter (sheath) from the carotid. A similar situation often arises when the stent delivery is attempted. Due to the lack of support, when axial forces are exerted on the tip, the above noted resistance to passage of devices into the carotid or recoiling of the guiding catheter (sheath) out of the common carotid occurs. This essentially denotes a “noncompliance” of either the aorta or the carotid with the inability of these arteries to conform to the applied axial force from the proximal end by the operator. The lack of “backup” support occurs because the guiding catheter (sheath) derives its backup support only from the contact it makes with the descending or transverse aorta. Because the surface area of contact between the secondary curve portion and the aortic wall is so small, the guiding catheter (sheath) is unstable and therefore easier to dislodge from its position against the aortic wall when resistive “pushback” forces are encountered during advancement of devices. The friction at these sites of contact is the limit to the axial force that can be conducted across the length of the guiding catheter (sheath). After the limit of this axial friction is overcome the guiding catheter (sheath) usually will recoil and buckle out of the carotid, endangering cerebral embolism.

This situation can be avoided by the deployment of our fixation device that would fixate the guiding catheter (sheath) to the transverse or descending aorta (FIGS. 2,3,4). This would allow transmission of the axial force along the length of the guiding catheter (sheath) without the risk of recoiling of the guiding catheter (sheath) out of the carotid artery if resistance is encountered. Thus it would facilitate passage of the embolic protection device, stent and the retrieval sheath into the carotid without any risk of recoiling of the guiding catheter (sheath) from the carotid and consequent movement of the embolic protection device and the associated risk of cerebral embolism.

### SUMMARY OF THE INVENTION

The present invention relates to a guiding catheter (sheath) that is specifically designed to facilitate the passage of therapeutic devices (embolic protection device, stent, retrieval sheath system) into the carotid artery. The present invention recognizes that the problem of backup support must be solved by making a fundamental change in the strategy the back up support is derived, by increasing the friction forces between the aorta and the guiding catheter (sheath).

The uniqueness of the guide catheter (sheath) of the present invention is the result of analysis of the factors determining the optimal support of a guide catheter within an aortic root complex and arranging these factors in a way to maximize backup support for distal advancement of a therapeutic device through the guide catheter. The catheter achieves a point of backup support against the wall of the descending or transverse aorta that is as close as possible to the ostium of the carotid artery without deriving any contact with the ascending aorta. This prevents any plaque to embolize from the ascending aorta that could predispose to occurrence of an cerebrovascular embolic event.

### BRIEF DESCRIPTION OF THE DRAWINGS

The figures illustrate the guiding catheter and its advantages with respect to allowing a stable cannulation of the carotid artery for undertaking carotid stenting.

FIG. 1 shows the positioning of the conventional guiding catheter (shuttle sheath) used for carotid intervention by telescoping it over a diagnostic carotid catheter-guidewire combination.

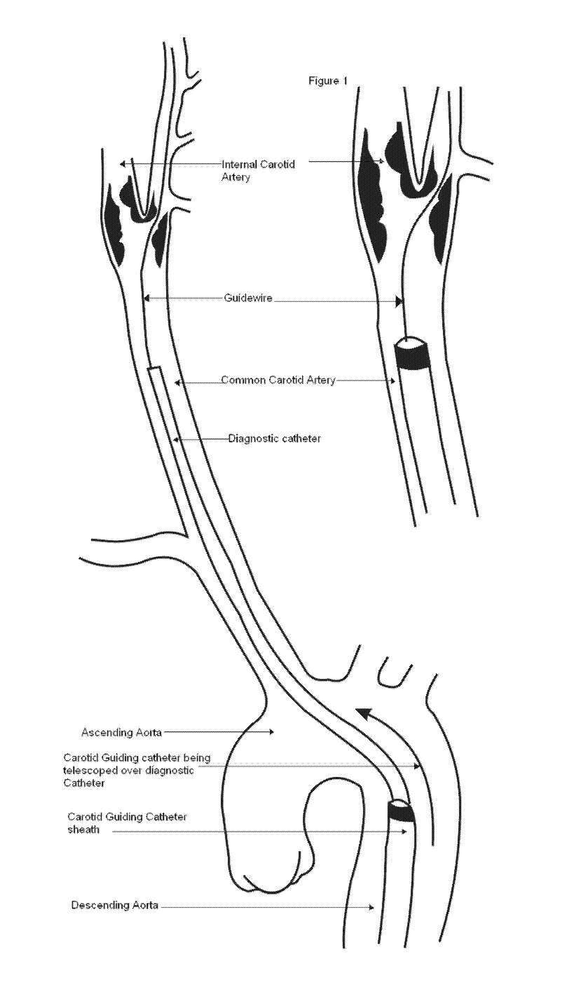

FIG. 2 shows the positioning of the new invention carotid guiding catheter. The collapsible anchoring wings have been deployed in the descending aorta, fixating the guiding catheter and allowing a stable position of the tip of the catheter in the common carotid artery to be achieved, preventing any axial movement of the guiding catheter in or out of the common carotid artery.

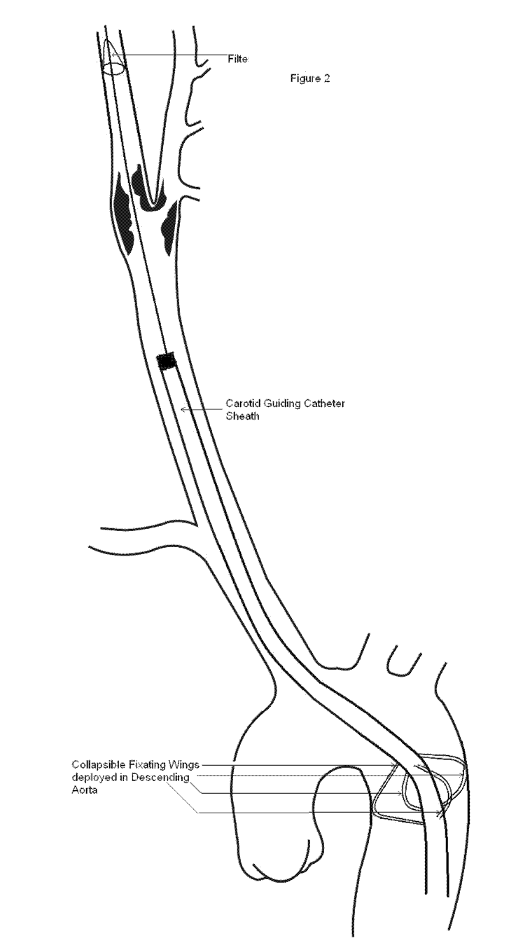

FIG. 3 shows an enlarged view of the new invention carotid guiding catheter positioned in the common carotid artery.

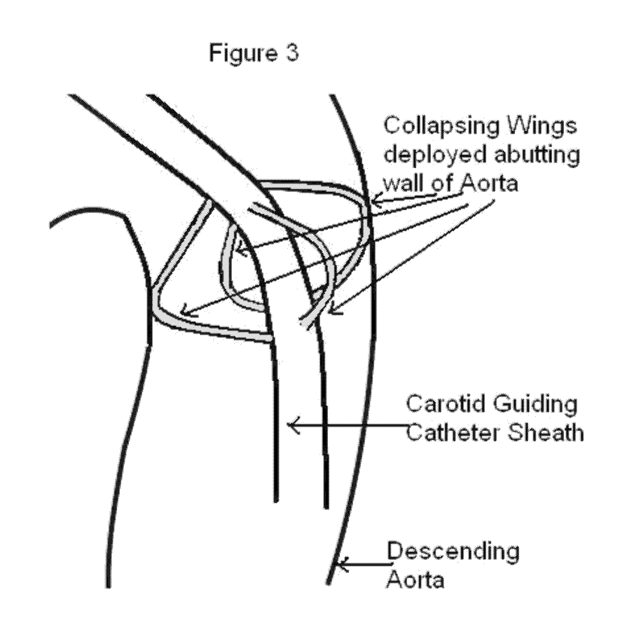

FIG. 4 shows the deployment of the collapsible anchoring wings in the descending aorta, fixating the new invention carotid guiding catheter against the wall of the aorta, thus providing a stable position of the new invention carotid guiding catheter in the common carotid artery.

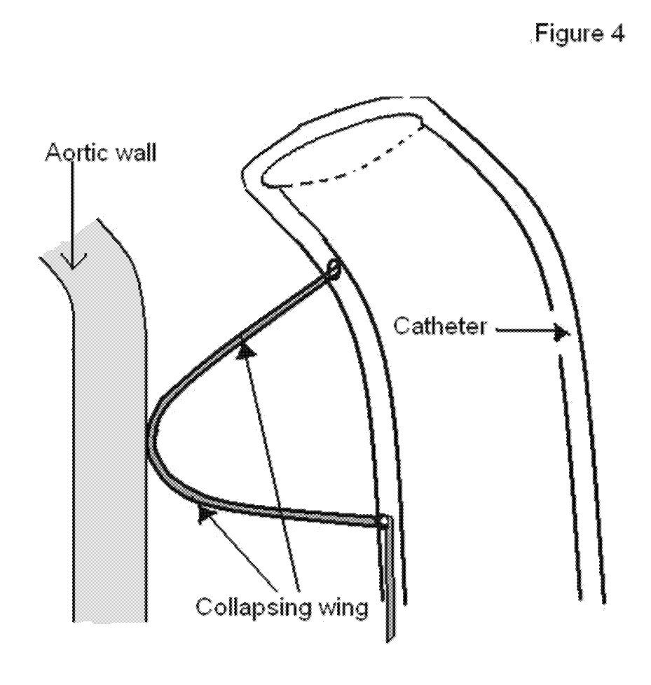

FIG. 5 is an enlarged cross-sectional view of the new invention carotid guiding catheter at the level of the collapsible anchoring wings showing the deployment of the collapsible anchoring wings, which are abutting the wall of the descending aorta.

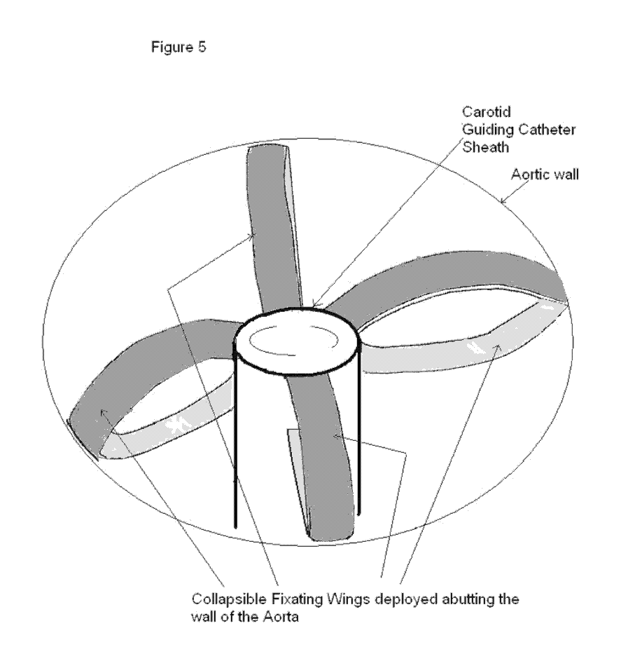

While the above identified drawings set forth several preferred embodiments, other embodiments of the present invention are also planned. This disclosure presents illustrative embodiments of the present invention by way of representation and not limitation. It should be understood that numerous other modifications and embodiments can be devised by those skilled in the art which fall within the scope and spirit of the principles of this invention.

### DESCRIPTION OF THE PREFERRED EMBODIMENTS

One embodiment of the catheter of the present invention is shown above. Other modifications, changes and substitutions are intended in the foregoing disclosure and in some instances some features of the invention will be employed without a corresponding use of other features. Accordingly, it is appropriate that the claims be construed broadly and in a manner consistent with the scope of the invention.

What is claimed is:

1. A guiding catheter (sheath) for use in treatment of carotid artery accessed from the aorta including descending aorta, aortic arch, ascending aorta. The guiding catheter sheath comprising:
a hollow, flexible tubular body having a proximal end and a distal end; and
means for engaging the tubular body with the wall of the ascending aorta , wall of the aortic arch or descending aorta when the distal end is generally coaxially aligned into the left or right common carotid artery; and
means for fixating the guiding catheter to the wall of the aorta with a fixating mechanism; and
means of deploying the fixating mechanism and collapsing the fixating mechanism
